(19) United States
(12) Patent Application Publication
(10) Pub. No.: US 2021/0398605 A1
(43) Pub. Date: Dec. 23, 2021

(54) SYSTEM AND METHOD FOR PROMOTER PREDICTION IN HUMAN GENOME

(71) Applicant: KING ABDULLAH UNIVERSITY OF SCIENCE AND TECHNOLOGY, Thuwal (SA)

(72) Inventors: Xin GAO, Thuwal (SA); Ramzan UMAROV, Thuwal (SA)

(21) Appl. No.: 17/297,233

(22) PCT Filed: Oct. 24, 2019
(86) PCT No.: PCT/IB2019/059139

§ 371 Date: May 26, 2021

Related U.S. Application Data
(60) Provisional application No. 62/774,494, filed on Dec. 3, 2018.

(51) Int. Cl. G16B 5/20; G16B 40/00; G06N 3/08; G06N 3/04

(52) U.S. Cl. CPC G16B 5/20; G06N 3/04; G06N 3/08; G16B 40/00

(57) ABSTRACT

A method for training a deep neural network model based on a known genome sequence includes receiving the known genome sequence; training the deep neural network model with a current negative set obtained from the known genome sequence; applying the deep neural network model to the known genome sequence and recording false positive sets; selecting a subset of the new false positive sets; updating the current negative set with the new false positive sets; and repeating the steps of training, applying, selecting and updating until a number of the new false positive sets is smaller than a given threshold.

### CROSS-REFERENCE TO RELATED APPLICATIONS

This application claims priority to U.S. Provisional Patent Application No. 62/774,494, filed on Dec. 3, 2018, entitled “DEERECT-PROMID: PROMOTER ANALYSIS AND PREDICTION IN THE HUMAN GENOME,” the disclosure of which is incorporated herein by reference in its entirety.

### BACKGROUND

### Technical Field

Embodiments of the subject matter disclosed herein generally relate to a system and method for analyzing and predicting a position of a promoter in the human genome, and more particularly, using sequence-based deep learning models that are trained based on an iterative approach that relies on false positives.

### Discussion of the Background

The high-fidelity of the RNA polymerase II (pol II) transcription system is necessary for precise spatiotemporal regulation of endogenous protein expression and essential to proper development and homeostasis in eukaryotes (an eukaryote is an organism whose cells have a nucleus enclosed within a membrane). Among the key cis-regulatory modules for RNA pol II-mediated transcription, there is a core promoter. The promoter is typically situated within a DNA segment spanning from −40 bp (base pairs) to +40 bp relative to a transcription start site (TSS), which is considered to be located at position+1 (see [1] and [2]). This segment of DNA serves as a platform on which the RNA pol II and a number of auxiliary factors assemble into the transcription machinery, which is capable of integrating a range of intrinsic and extrinsic signals, to ultimately determine the proper initiation of DNA transcription. Thus, the characterization of the structure-function relation of the core promoter is necessary to be known for unraveling the complex molecular control mechanisms underlying not just the constitutive basal expression, but also the regulated expression in the RNA pol II transcription system.

Past in vitro research has identified a number of functional sequence motifs for the RNA pol II core promoter. Among such functional core promoter elements, perhaps, the most well-known is the TATA box (the TATA box is considered a non-coding DNA sequence, also known as a cis-regulatory element, which contains a consensus sequence characterized by repeating T and A base pairs), which was, in the past, thought to be universally present in the RNA pol II core promoters. However, the advent in genome-wide TSS detections based on high-throughput sequencing revealed that the core promoter structure is highly diverse and complex, and there are no universal core promoter elements. Indeed, recent estimates showed that only about 17% of eukaryotic core promoters contain the TATA box.

In fact, genome-wide structural analysis found that many core promoters do not possess any of the known core promoter elements. Such structural heterogeneity permits the core promoter to expand its functional repertories so as to serve as gene- and cell-type-specific transcription regulator that responds to a range of conditions. However, because of this large diversity, the design principles of the core promoter still remains largely elusive and thus, the core promoter's location is difficult to find in the genome.

The structure of the human promoter is notoriously complex and diverse. One explanation for this is that such complex and diverse structures must be “designed” to properly control expression of about 25,000 protein coding genes based on interactions with only about 1,850 transcription factors in the human genome. Another explanation is based on a molecular evolution study which discovered substantially accelerated rates of evolution in primate promoters compared with other mammalian promoters. This rapid primate promoter evolution was found to be comparable to the neutral substitution rate, suggesting that primate promoters have weak selective constraints, and this suggestion can also explain highly-complex and diverse structures in the human promoter.

A better understanding of the structure-function relation of the human promoter has particularly important implications as some genetic variants in such noncoding regions are associated with rare Mendelian diseases. Further, some cancer cells are associated with somatic mutations in the promoter regions. In order to gain insights into what types of genetic variations can cause aberrant expression leading to human diseases, it is important to accurately predict the locations of human promoters in the genome and to understand their structural patterns.

As the existing methods are not very accurate in determining the locations of the core promoters, there is a need for a new promoter detection method that more accurately identifies the location of the core promoter in the genome.

### BRIEF SUMMARY OF THE INVENTION

According to an embodiment, there is a method for training a deep neural network model based on a known genome sequence. The method includes receiving the known genome sequence, training the deep neural network model with a current negative set obtained from the known genome sequence, applying the deep neural network model to the known genome sequence and recording false positive sets, selecting a subset of the new false positive sets, updating the current negative set with the new false positive sets, and repeating the steps of training, applying, selecting and updating until a number of the new false positive sets is smaller than a given threshold.

According to another embodiment, there is a method for determining a transcription start site of a promoter in a genome sequence. The method includes receiving a genome sequence, training a deep neural network model based on an interactive and adaptive approach that updates a current negative set based on determined false positives, applying the genome sequence to the deep neural network model, and determining the transcription start site of the promoter in the genome sequence based on the updated current negative set.

According to still another embodiment, there is a computing device that implements a deep neural network model, and the device includes a processor having an input layer configured to receive a known genome sequence and plural convolutional neural networks, CNN, layers, each connected to the input layer, and configured to train with a current negative set obtained from the known genome sequence, a memory connected to the processor and configured to record false positive sets when the deep neural network model is applied to the known genome sequence, and the processor is configured to select a subset of the new false positive sets, to update the current negative set with the new false positive sets, and to repeat the steps of training, applying, selecting and updating until a number of the new false positive sets is smaller than a given threshold.

### BRIEF DESCRIPTION OF THE DRAWINGS

For a more complete understanding of the present invention, reference is now made to the following descriptions taken in conjunction with the accompanying drawings, in which:

FIG. 1 is a schematic diagram of a deep neural network model used to determine a transcription start site for a promoter in a genome sequence;

FIGS. 2A and 2B illustrate the scoring landscape for promoters with a TATA box obtained by using the deep neural network model of FIG. 1;

FIGS. 3A and 3B illustrate the scoring landscape for promoters without a TATA box obtained by using the deep neural network model of FIG. 1;

FIG. 4 is a flowchart of a method for determining the transcription start site for the promoter in the genome sequence;

FIG. 5 schematically illustrates the method for determining the transcription start site for the promoter in the genome sequence;

FIG. 6 illustrates the influence of different regions inside the promoter on the score produced by the deep neural network model of FIG. 1;

FIG. 7 presents results obtained with the deep neural network model of FIG. 1 and traditional methods for determining the transcription start site for the promoter in the genome sequence;

FIG. 8 illustrates the promoters predicted by the method of FIG. 4 and traditional methods for a given DNA sequence;

FIG. 9 is a sequence logo of the most important 15-mers identified by the deep neural network model of FIG. 1;

FIG. 10 is a sequence logo around a known transcription start site for a promoter;

FIG. 11 is a flowchart of a method for training the deep neural network model of FIG. 1;

FIG. 12 is a flowchart of a method for finding the transcription start site of a promoter with a model trained as discussed in FIG. 11; and

FIG. 13 is a schematic diagram of a computing device that implements the deep neural network model of FIG. 1.

### DETAILED DESCRIPTION OF THE INVENTION

The following description of the embodiments refers to the accompanying drawings. The same reference numbers in different drawings identify the same or similar elements. The following detailed description does not limit the invention. Instead, the scope of the invention is defined by the appended claims.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to an embodiment, a novel machine learning-based approach for the prediction of human RNA pol II core promoters, called herein PromID, is introduced. Taking advantage of the big promoter collection with experimentally validated TSSs generated by modern high-throughput techniques, PromID builds a deep learning model using sequence data as its input. To avoid bias based on prior knowledge about the promoter loci (e.g., sequences with known core promoter elements and high-density of CG dinucleotides), the PromID method does not use predefined features, but rather attempts to discover sequence features and learn salient patterns of the human promoter solely from the training set. This feature of the method distinguishes this approach from the existing methods especially in the prediction of human promoters, because the structural features of many promoters are still unknown.

The inventors of the PromID method have previously developed a convolutional neural network-based algorithm for the prediction of core promoter locations in several model organisms [3]. While this method was able to outperform previously developed promoter prediction methods, its false positive rate is not good enough to ensure the accurate detection of promoters on long genomic sequences. The PromID method alleviates this limitation and focuses more on the promoter prediction on longer sequences. In one embodiment, to reduce the false positive rate, the PromID method adaptively and iteratively trains the predictor by changing the distribution of samples in the training set based on the false-positive errors it made in the previous iteration. By increasing the weight of difficult non-promoter sequences in the training set, the method forces the predictor to learn promoter patterns to rule out such sequences.

To evaluate the performance of the novel PromID method, comparisons of the neural network model with publicly available tools for the human promoter prediction task are later presented. It was found by the inventors that the PromID method outperformed the other predictors and achieved a much smaller error-per-1,000-bp rate than the others. These results, which are presented later, demonstrate the usefulness of the PromID method for the human promoter prediction on long genomic sequences and suggest its potential value as a tool to gain insights into the design principle for the human core promoters.

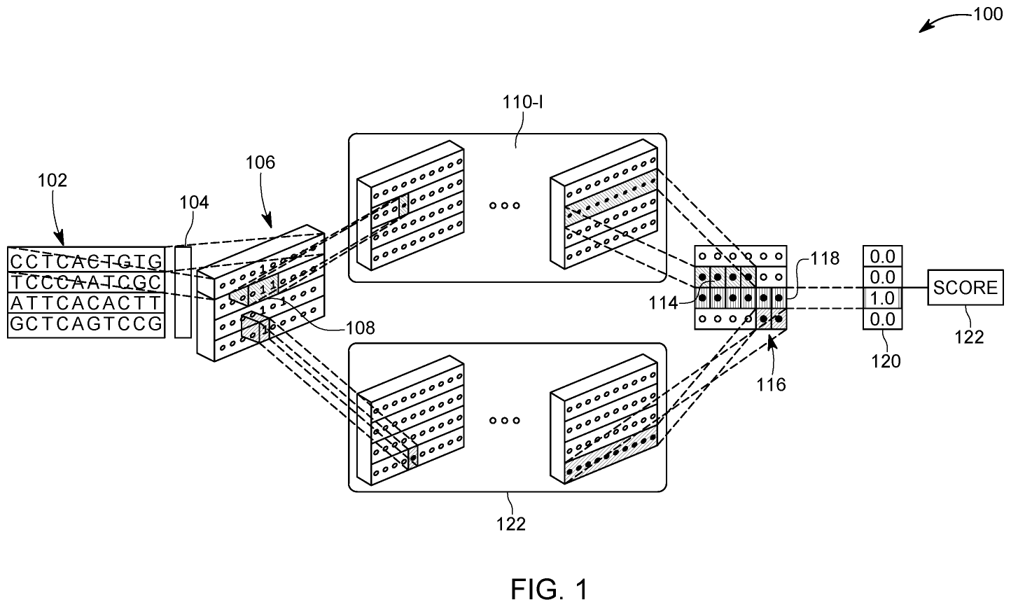

A deep neural network model 100, as illustrated in FIG. 1, is used as the engine for the PromID method. The deep neural network model 100 is used to distinguish the promoters from the non-promoters. The deep neural network model 100 receives DNA sequences 102 at an input layer 104. The input data is read in the fasta format and then encoded using a one hot encoding layer 106. This encoding procedure uses a vector of size 4 to represent each nucleotide A, T, G, and C. For example, A is encoded as (1 0 0 0), T is encoded as (0 1 0 0), G is encoded as (0 0 1 0), and C is encoded as (0 0 0 1).

The encoded data 108 is distributed to plural Convolutional Neural Networks (CNN) layers 110-I, where I is an integer between 1 and 10. The CNN layers 110-I are configured to work in parallel, as illustrated in the figure. This means that each CNN layer 110-I has direct access to the encoded data 108. Each CNN layer 110-I has a filter and the filter lengths for the various CNN layers are different, each CNN having a unique filter length so that these filters are able to represent different promoter elements. In one application, the PromID method uses a special CNN layer 112 having a filter length of just one. The special CNN layer 112 with filter length one is able to capture the GC content of the genome sequence 102. The GC content of the genome sequence is known to be higher in a promoter region than in another region of the DNA. While the CNN layers 110-I use a maximum pooling, the special CNN layer 112 is the only one using an average pooling because the method is interested about the count of the G and C nucleotides, and not about their positions inside the promoter. In this regard, pooling layers are used to streamline the underlying computation. Pooling layers reduce the dimensions of the data by combining the outputs of the neuron clusters at one layer into a single neuron in the next layer. The pooling may compute a max or an average. Max pooling uses the maximum value from each of a cluster of neurons at the prior layer while the Average pooling uses the average value from each of a cluster of neurons at the prior layer.

The output 114 from the CNN layers 110-I and 112 is then concatenated and flattened with a concatenation layer 116 and the concatenated and flattened data 118 is then fed to a softmax layer 120. Note that model 100 does not have a fully connected dense layer as it was observed by the inventors that such a layer would actually reduce the predictive power of the model. Although each individual layer of the model 100 is known in the art, the combination of the layers illustrated in FIG. 1 is novel.

The softmax layer 120 has two neurons which represent an input sequence being a promoter (pp) or a non-promoter (pnp). The final score 122 produced by the model 100 is calculated as follows:

$$Score = \frac{( {p}_{p} - {p}_{np} + 1 )}{2} . \qquad ( 1 )$$

This score has values in the range from 0 to 1 and is used as a proxy for the probability that an input sequence is a promoter.

In one application, the PromID method uses a weight decay and dropout to improve the generalization capability of the model. An Adam optimization algorithm may be used to train the weights [4], which is an improved version of stochastic gradient descent. In one implementation, the TensorFlow (Abadi et al., Tensorflow: a system for large-scale machine learning, OSDI, volume 16, pages 265-283, 2016) is used as the framework to construct the deep neural network.

The model 100 was trained using human promoter sequences extracted from the EPDnew database. The EPD database is an annotated non-redundant collection of eukaryotic POL II promoters, for which the transcription start site has been determined experimentally. The authors of the EPDnew database have demonstrated its higher quality over the ENSEMBL-derived human promoter set. For the training, the inventors downloaded 16,455 genomic sequences (from −5000 bp to +5000 bp, where +1 is a TSS position) containing human promoters from the EPD database. In one embodiment, 90% of the downloaded sequences were used for training and 10% were used for testing. Positive and negative sets were extracted from the training set. A promoter region of a given size around the known TSS is considered herein to be a positive sequence. A negative sequence is considered to be a region outside the promoter region, which does not contain a known TSS. Initially, the negative set had the same size as the positive one and consisted of randomly picked sequences.

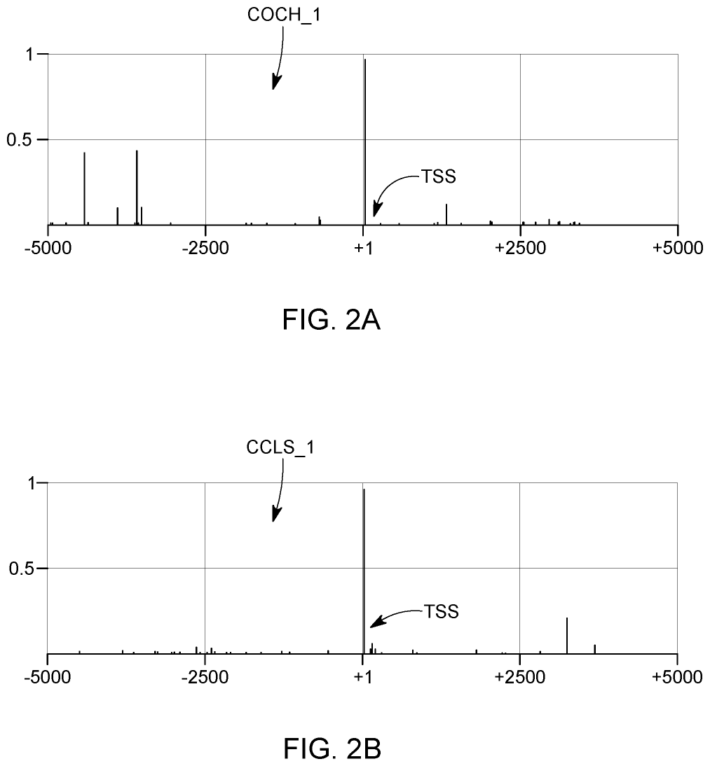

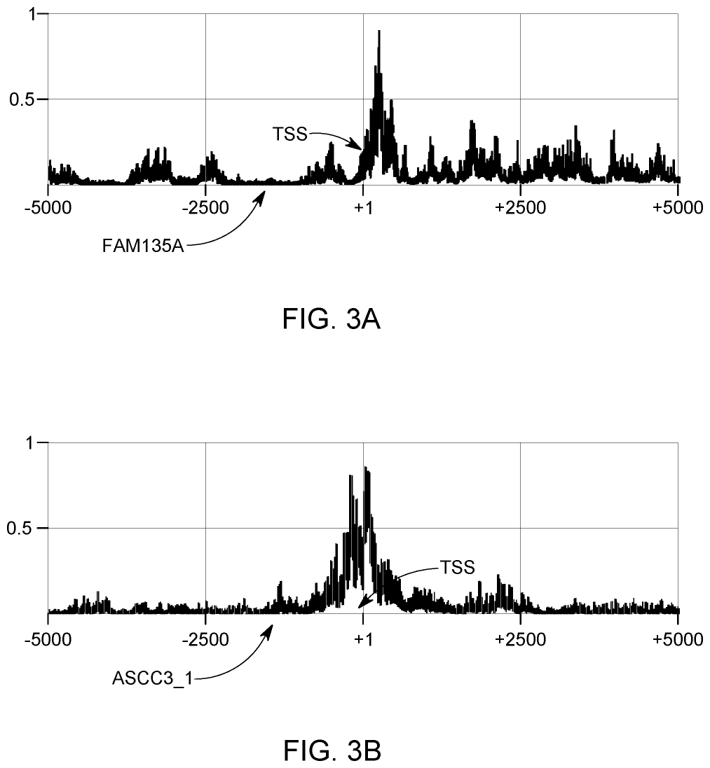

The model 100 was trained using positive and negative sets, which include relatively short sequences with a fixed length. As the model 100 accepts sequences of certain length as input, a sliding window approach was taken to analyze long genomic sequences. This window is moved across the sequence and at each predefined position, the subsequence in the window is fed to the model 100. For this reason, FIG. 1 show plural DNA sequences 102 as being the input. The model gives a score 122 from 0 to 1, based on equation (1), to each sequence. The score represents the likelihood that an input subsequence includes a promoter region. If these scores are plotted, a scoring landscape for the model is obtained, as illustrated in FIGS. 2A to 3B. Note that each of these figures show a highest score at the TSS position, and the genes having the promoter regions were COCH_1 in FIG. 2A, CCL5_1 in FIG. 2B, FAM134A_1 in FIG. 3A, and ASCC3_1 in FIG. 3B.

If the value of the score of a sliding window is above a set threshold, then that position is predicted to be a start of the promoter region. In one embodiment, the method uses two deep learning models (each similar to model 100)—one for the identification of promoter sequences having the TATA box and one for promoters without the TATA box. Note that the promoters can be predicted more accurately if they have the TATA box, and that is the reason why the model 100 was first trained specifically for the promoters with the TATA box (called herein the TATA+ model). Next, the model was trained with the promoter sequences without the TATA box (called herein the TATA− model). The predictions of the second model, TATA− model, which are not too close to the first model TATA+ model predictions, are considered. For example, in one application, the TATA− model predictions are required to be at least 1,000 bp apart from the predictions of the TATA+ model. The two sets of predictions are then combined to make a final decision about the position of the promoter region. TSS is then considered to be at a certain position inside the promoter region. For example, if the sliding window has a 600 bp length and the positive set was extracted from −200 bp to +400 bp, then the TSS will be located at position 201 inside the predicted promoter region.

When constructing the prediction model to classify the promoters, it is necessary to choose what sequences to use for the non-promoter regions, i.e., for the negative sets. This problem is important because it affects what features the model will use to separate the two classes of positive and negative sets. For example, suppose that random DNA sequences are selected, which do not include promoters, for the negative set. In this case, a very small number of them will have the TATA motif at the specific position. Then the neural network model will just use this one feature to achieve almost perfect separation between the two classes. When applying such a model to the real world data, although the sensitivity will be high, this model will generate many false positives. Any sequence with a TATA motif at the specific position will most likely be classified as a promoter. Simply increasing the negative set size is not an effective solution as well, because the data becomes unbalanced and also, there will be a high chance that neural networks will be stuck at some local minimum as in the case considered above. There are not many sequences in the negative set that will have a good scoring TATA motif, which will make the model likely to distinguish the various sets heavily based on this single discriminating feature.

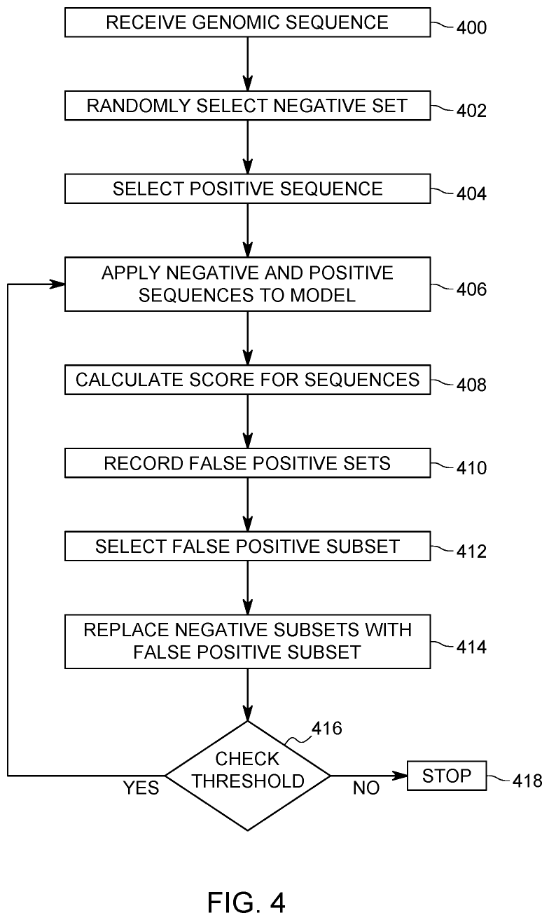

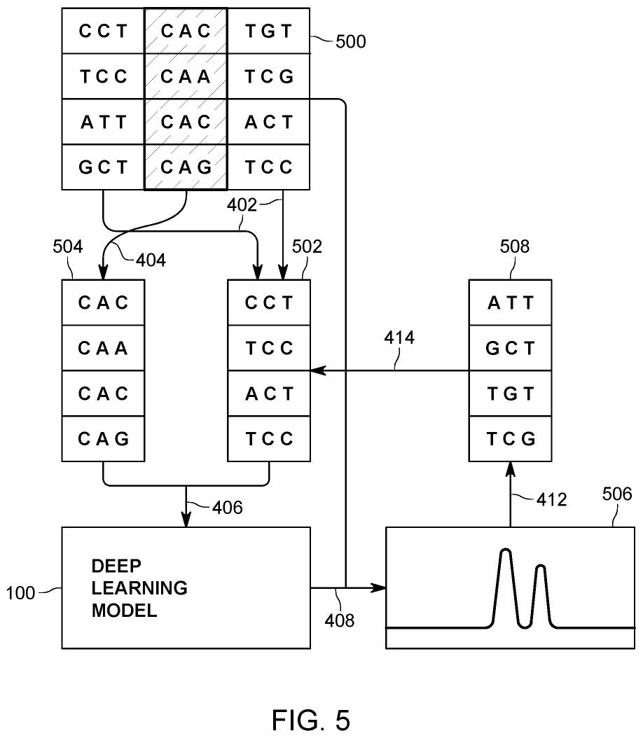

The inventors have found that an iterative and adaptive training approach for the model 100 resolve these issues. According to this approach, which is illustrated in FIGS. 4 and 5, in step 400 a genomic sequence 500 is received. In step 402, a negative set 502 is randomly selected from the genomic sequence 500 and in step 404 a positive sequence is selected. In step 406, the positive set 504 and the negative set 502 are applied to the model 100 for training. Note that the model 100 is trained with the current negative set 502. However, the current negative set 502 changes from iteration to iteration, as discussed later, based on results from a previous iteration, which makes the method adaptive. A score 506 for each window of the sequence is calculated in step 408. The false positive sets are recorded in step 410. Note that the false positive sets can be determined because the TSS positions for the actual promoters are known for the selected training data.

A subset 508 of the false positive sets having the highest scores calculated by the model 100 in step 408, i.e., the ones that are most similar to the true promoters from each long sequence 102, is chosen in step 412 as part of the new negative set 502. The current negative set 502 is then updated in step 414 by merging part of the previous negative set 502 with the subset 508. For example, the subset 508 may include any number of false positives between 1 and the maximum number of false positives. The part of the previous negative sets that are kept in the new negative sets may be about half of the originally randomly selected negative sets. For example, if the original negative sets are 20,000, which were randomly selected, 10,000 of them are removed in step 414 and replaced by the new subset 508 of false positives that have the highest score 506. One skilled in the art would understand that the number of negative sets removed from the old negative sets may be less or more than half of the original negative sets.

Then, in step 416, the method compares the number of newly found false positives with a threshold number, and if this number is larger than the threshold, the method returns to step 406. If the number of newly found false positives is smaller than the threshold, than the method stops in step 418 and the model 100 is trained and ready to be applied to a genomic sequence that has unknown promoters. By replacing some of the known negative sets with the false positives, the method of FIG. 4 constructs a difficult negative set, which forces the neural network to learn deeper and less obvious features to recognize a promoter sequence.

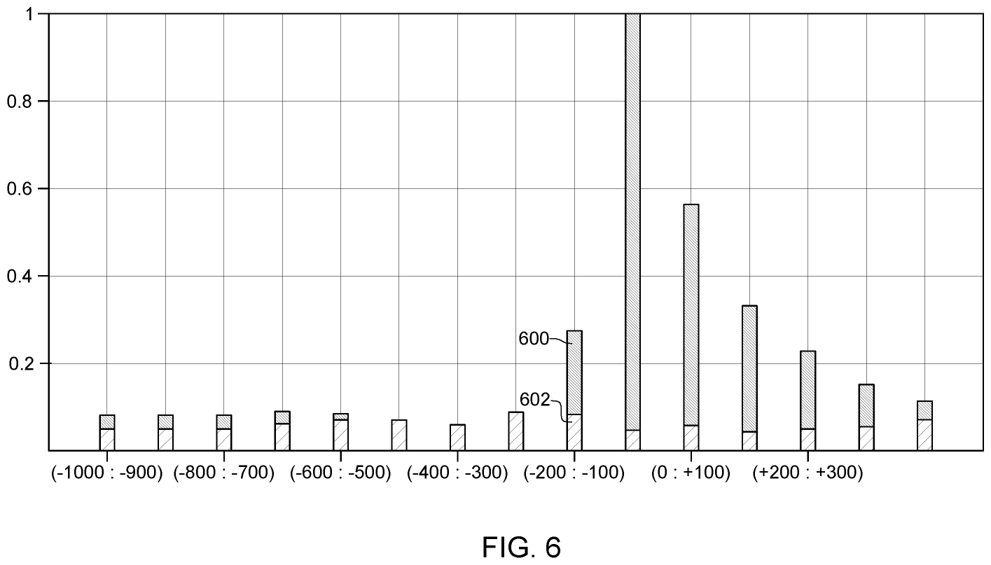

In previous work performed on promoter identification, the inventors used a region from −200 bp to +50 bp to extract promoter features. Because multiple transcription start points often significantly enlarge the potential gene promoter regions, the method illustrated in FIGS. 4 and 5 was configured to have a promoter model that uses a much wider region, from −1000 bp to +500 bp, and then apply a random substitution procedure to study the location of the sequence elements affecting the promoter prediction performance and potentially narrow the region down. The random substitution procedure works as follows. Suppose there is a window of size 100, which is moved along each sequence with a step size of 100. At each position, the random substitution procedure replaces the nucleotides within the window with 100 random nucleotides and calculates a new promoter score for the modified sequence. The difference between the original score and the new score is recorded and reported for each position, as illustrated in FIG. 6. Note that part 600 represents the decrease of the score after the random substitution and part 602 represents the increase of the score after the random substitution. It is noticed that the region from −200 bp to +400 bp has the most significant effect on the score predicted by the model 100 and this is why this procedure was implemented to select the size of the region used to train the final model.

To evaluate the accuracy of the model 100 trained based on the method illustrated in FIG. 4, and to objectively compare the predictions of the model 100 and other promoter identification methods, the performance of the model was measured using the Recall, Precision, and Correlation Coefficients (CC) as follow:

$$\begin{matrix}Recall = \frac{TP}{TP + FN} , & ( 2 ) \\ Precision = \frac{TP}{TP + FP} , & ( 3 ) \\ CC = \frac{TP \times TN - FP \times FN}{\sqrt{( TP + FP ) ( TP + FN ) ( TN + FP ) ( TN + FN )}} , & ( 4 )\end{matrix}$$

where TP is true positive, FN is false negative, and FP is false positive.

If the trained model 100 predicts a promoter with the TSS, which is closer to the known TSS by the allowed margin for error (500 bp), then this prediction is counted as a TP. If there is no prediction in the area from −500 bp to +500 bp of the known TSS, then this event is counted as a FN. Any prediction outside the region from −500 bp to +500 bp of some TSS is counted as a FP. The same rule is applied for performance evaluation to all the other tested promoter prediction programs. In addition, the inventors used two accuracy measures that are useful to evaluate the performance of the promoter prediction tools when analyzing long genomic sequences: the average prediction error per correctly predicted TSS, and the average prediction error per 1,000 bp.

The trained model 100 was compared with TSSW (Salamov and Solovyev, The gene-finder computer tools for analysis of human and model organisms genome sequences. In Proceedings of the Fifth International Conference on Intelligent Systems for Molecular Biology, AAAI Press, Halkidiki, Greece, pages 294-302, 1997), which uses a linear discriminant function combining a TATA box score, triplet preferences around the TSS, hexamer preferences and potential transcription factor binding sites. The TSSW has shown good results. Another existing tool is the FPROM, which was created by extending the TSSW program feature set, which resulted in significant improvement over TSSW and other promoter recognition software (Solovyev and Shahmuradov, Promh: promoters identification using orthologous genomic sequences. Nucleic acids research, 31(13):3540-3545, 2003). Still another promoter prediction tool is the Promoter 2.0 (Knudsen, Promoter 2.0: for the recognition of polii promoter sequences. Bioinformatics (Oxford, England), 15(5):356-361, 1999. 1999), which extracted promoter elements from DNA sequences and used artificial neural network (ANN) to distinguish promoters from non-promoters based on these features. Yet another tool is DragonGSF (Bajic and Seah, Dragon gene start finder: an advanced system for finding approximate locations of the start of gene transcriptional units. Genome research, 13(8):1923-1929, 2003), which also used ANN as a part of its design and considered the GC content and the concept of CpG islands for promoter recognition.

The inventors' previous promoter recognition software, PromCNN achieved good classification performance in discriminating between short promoter and non-promoter sequences [3]. Recently PromCNN was outperformed by (Qian et al., An improved promoter recognition model using convolutional neural network. In 2018 IEEE 42nd Annual Computer Software and Applications Conference (COMPSAC), pages 471-476, IEEE, 2018), which achieved an improved accuracy by about 7%. However, as in [3], this approach focused on the classification performance of short sequences, instead of promoter identification for a long genomic sequence. The latter is a much more difficult problem to tackle because of the high risk of having a large number of false positives. The results of the new method illustrated in FIG. 4 was not compared to the results of the model in Qian et al. because this group did not provide a web server or a tool that would accept long genomic sequences as inputs.

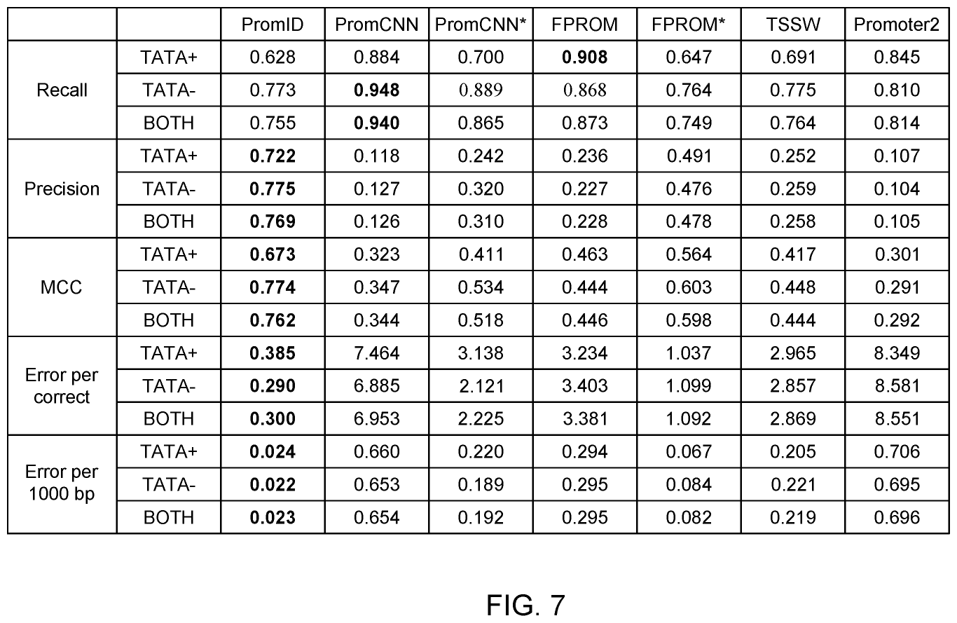

The table shown in FIG. 7 presents the results of the comparison of the method of FIG. 4 with the following methods: PromCNN, TSSW, FPROM, and Promoter 2.0. It is noted that all these programs have high recall and mostly very low precision. Thus, the inventors have modified the parameters of these models to reduce the number of false positives. This was beneficial to FPROM, for which the MCC increased from 0.446 to 0.598 and to PromCNN, for which the MCC was increased by 0.174, but not for the other models.

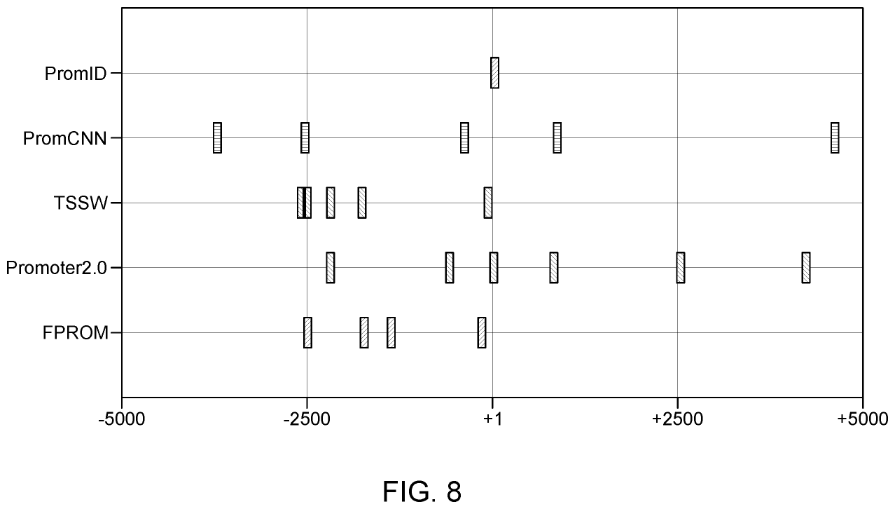

Regardless of the tested parameters, the PromID method significantly outperforms the other examined methods. For example, the PromID method using the model 100 trained on the [−200 +400] region has a precision and MCC higher than the best competing tool, FPROM, by 0.291 and 0.164, respectively, for the similar recall of 0.749. In this regard, FIG. 8 shows an example of the predictions made by the different promoter prediction programs on the sequence containing the promoter of the UBE3D_1 gene. It can be seen that the PromID method makes no false positive predictions (a false positive prediction is any promoter that is predicted at a position different from +1) while still successfully finding the true TSS for this gene, while all the other models make plural false positive predictions.

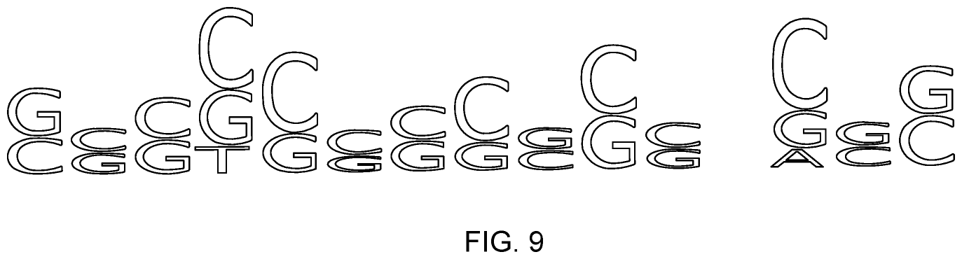

It is known in the art that the models trained by neural networks are difficult to interpret. The inventors propose to overcome this limitation by visualizing the trained convolutional filters. As the maximum filter length used in one embodiment is 15, it was decided to find the most important 15-mers identified by the model 100. The top 1,000 most influential 15-mers were identified with this model and a sequence logo was built for them as illustrated in FIG. 9. The top three most important motifs were found to be CCCAGGACCATGTCT, GCTAGGTTGTTATGT, GTTCCCGGCCGGTGC, which all contain GC rich subsequences that are well known characteristics of the eukaryotic promoters. Note that at some of the positions in the 15-mers there are also A and T nucleotides, but because their content for the 1,000-mers is very small, the A and T nucleotides are not present at those positions in FIG. 9. The relative sizes of the letters C, G, T, and A in FIG. 9 indicate their frequency in the sequences while the total height of the letters indicates the information content of the position, in bits.

To see the contributions of different nucleotides at different positions of the promoter sequences, the inventors used a modification of the so called feature mutation map for all sequences in the test set. The mutation maps for TATA+ and TATA− promoters were built by taking a set of genomic non-promoter sequences with sizes equal to the input sequences used in the promoter models and studied how nucleotide substitutions changes the promoter score computed by the TATA+ and TATA− models. At each position of the tested sequences, a nucleotide was replaced with a different one in all these sequences and their average promoter score was computed. The mutation maps show significant differences of sequence features of TATA+ and TATA− promoters and the location of their most conserved elements.

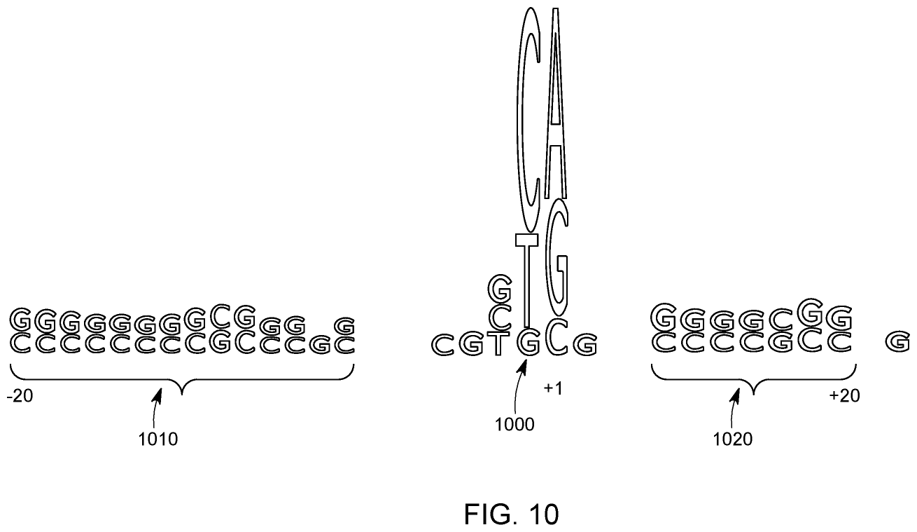

It was also observed that the largest effect on the score in the TATA+ model comes from the T/A-rich TATA− box region. The most significant element of the TATA− promoters is the initiator element, which is very similar to the new consensus sequence for the human initiator (Inr) core promoter element BBCABW (where B=C/G/T and W=A/T). Such initiator element typically directs the positioning of the transcription initiation start sites representing so called focused promoters in which the transcription initiates at a single site or a narrow cluster of sites. The initiator element contains a conserved motif, which is observed in both (TATA+ and TATA−) data sets in FIG. 10. FIG. 10 is a sequence logo of the region from −40 bp to +40 bp around the known TSS. The sequence logo demonstrates the conservation in the promoter initiator region 1000 as well as in two GC rich regions 1010 and 1020 upstream and downstream from the TSS.

For the TSS position (+1) in FIG. 10, the most preferred nucleotides are A and G. If a promoter has the initiator element then A is the most frequent nucleotide at position+1, otherwise it is G. The sequences at positions −1 to +3 are the most important for setting the levels of the basal transcription. Changing the nucleotides in the region −30 bp to −23 bp from the original ones to G or C reduces the score considerably. While the promoter regions, in general, have more G and C nucleotides, the mentioned region contains the TATA box in the TATA+ model and tends to have T/A nucleotides in TATA− promoters, which is why setting various nucleotides to G or C has a negative effect on the score. For the TATA− promoters, it was observed the occurrence of GC rich elements, which is in agreement with the findings in FIG. 9 that the most significant promoter 15-mers are GC-rich.

As have been discussed before, promoters with the TATA box can be predicted with a very small positional error. Often the predicted TSS is exactly at the position of the true TSS. High positional accuracy of the TATA promoters is the result of the conserved motif fixating position of the promoter region. However, for the promoters without the TATA box is not the same and for these promoters the predicted positions have a normal distribution around the true TSS. For about 15% of sequences in the test set, the predicted TSSs are further than 100 bp from a true TSS. This problem can be partially explained by the occurrence of multiple TSSs in non-TATA promoters. Such promoters generate alternative gene isoforms that have tissue or time specific expression. It was shown in the literature that promoters have focused, dispersed, and mixed transcription. For dispersed transcription, there are many weak TSSs located in the region from −50 bp to +50 bp. These multiple transcription start sites might be responsible for a wide promoter score peak (see FIGS. 3A and 3B) for non-TATA promoters generated by the deep learning model. Many of such multiple TSSs as well as some distant alternative TSSs are not annotated in the promoter databases and currently they are considered as false positives predictions while their actual status requires further experimental verification.

The model 100 introduced above in FIG. 1 and its training method introduced in FIG. 4 show one or more advantages over the existing models. In this regard, while previously developed promoter prediction methods can relatively accurately classify promoter and non-promoter sequences, they fail to provide good results when applied to long genomic sequences. Due to the potentially large amount of tested locations, all these traditional methods have very low precision and generate a large number of false positives (often much more than the number of real promoters), which limits their usage in genome-scale studies.

The model 100 and its training method overcome these issues by using an iterative and adaptive training approach that focuses on instances that were misclassified by previous iterations and builds the deep learning model to be able to eliminate the huge number of false positives. Comparisons of the model 100's performance with the available promoter prediction tools demonstrate that the PromID method significantly outperforms the others. Because many genes have non-coding exons and the traditional gene-finders cannot provide the actual gene start and promoter position, programs that accurately perform computational identification of promoters are important for revealing the gene structure and studying gene regulation. The model and training method discussed herein contributes towards this goal.

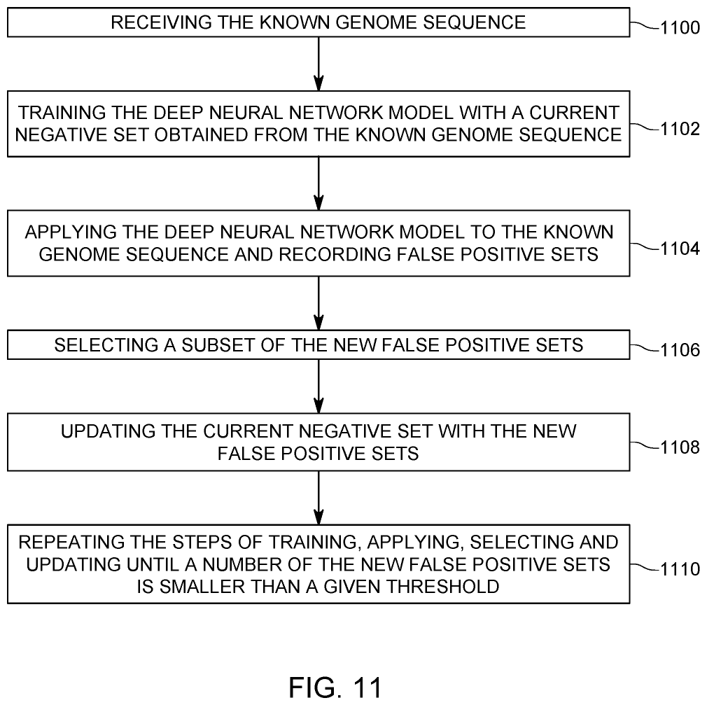

A method for training a deep neural network model based on a known genome sequence is now discussed with regard to FIG. 11. The method includes a step 1100 of receiving the known genome sequence, a step 1102 of training the deep neural network model with a current negative set obtained from the known genome sequence, a step 1104 of applying the deep neural network model to the known genome sequence and recording false positive sets, a step 1106 of selecting a subset of the new false positive sets, a step 1108 of updating the current negative set with the new false positive sets, and a step 1110 of repeating the steps of training, applying, selecting and updating until a number of the new false positive sets is smaller than a given threshold.

A negative set includes a region of the known genome sequence that does not include a promoter, a positive set includes a region of the known genome sequence that includes a promoter, and a false positive set includes a region of the known genome sequence that does not include a promoter but is found by the deep neural network model to correspond to a promoter.

The method may further include a step of calculating a score for plural sets of the known genome sequence based on plural convolutional neural networks layers of the deep neural network model, and a step of selecting the subset of the new false positive sets based on a highest score of the plural sets. In one application, the score is calculated by a softmax layer, the softmax layer has two neurons, a first neuron which represents an input sequence being a promoter (pp) and a second neuron which represents an input sequence being a non-promoter (pnp). The score may be given by a difference of pp and pnp, to which the unity is added, and the result is divided by 2.

In one embodiment, the step of updating further includes removing a subset of the current negative set. The method may further include a step of training the deep neural network model for promoters having a TATA box to obtain a TATA+ trained model, and a step of training the deep neural network model for promoters not having a TATA box to obtain a TATA− trained model. The current negative set is originally randomly obtained from the known genome sequence.

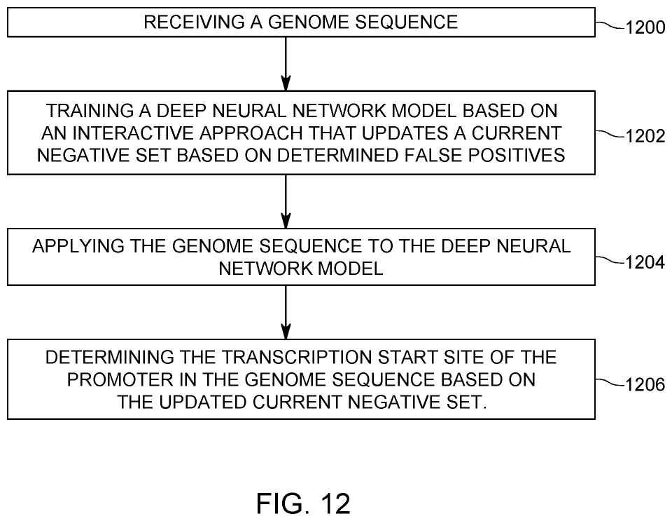

A method for determining a transcription start site of a promoter in a genome sequence is now discussed with regard to FIG. 12. The method includes a step 1200 of receiving a genome sequence, a step 1202 of training a deep neural network model based on an interactive approach that updates a current negative set based on determined false positives, a step 1204 of applying the genome sequence to the deep neural network model, and a step 1206 of determining the transcription start site of the promoter in the genome sequence based on the updated current negative set. In one application, the training step includes training the deep neural network model with a current negative set obtained from a known genome sequence, applying the deep neural network model to the known genome sequence and recording false positive sets, selecting a subset of the new false positive sets, updating the current negative set with the new false positive sets, and repeating the steps of training, applying, selecting and updating until a number of the new false positive sets is smaller than a given threshold.

A negative set includes a region of the known genome sequence that does not include a promoter, a positive set includes a region of the known genome sequence that includes a promoter, and a false positive set includes a region of the known genome sequence that does not include a promoter but is found by the deep neural network model to correspond to a promoter.

The method may further include a step of calculating a score for plural sets of the known genome sequence based on plural convolutional neural networks layers of the deep neural network model, and a step of selecting the subset of the new false positive sets based on a highest score of the plural sets. The score is calculated by a softmax layer, the softmax layer has two neurons, a first neuron which represents an input sequence being a promoter (pp) and a second neuron which represents an input sequence being a non-promoter (pnp). In one application, the score is given by a difference of pp and pnp, to which the unity is added, and the result is divided by 2.

The step of updating may further include a step of removing a subset of the current negative set. The method may also include a step of training the deep neural network model for promoters having a TATA box to obtain a TATA+ trained model, predicting promoters having the TATA box by using the TATA+ trained model, training the deep neural network model for promoters not having a TATA box to obtain a TATA− trained model, predicting promoters not having the TATA box by using the TATA− trained model, and combining the promoters having the TATA box with the promoters not having the TATA box to determine the transcription start site of the promoter in the genome sequence.

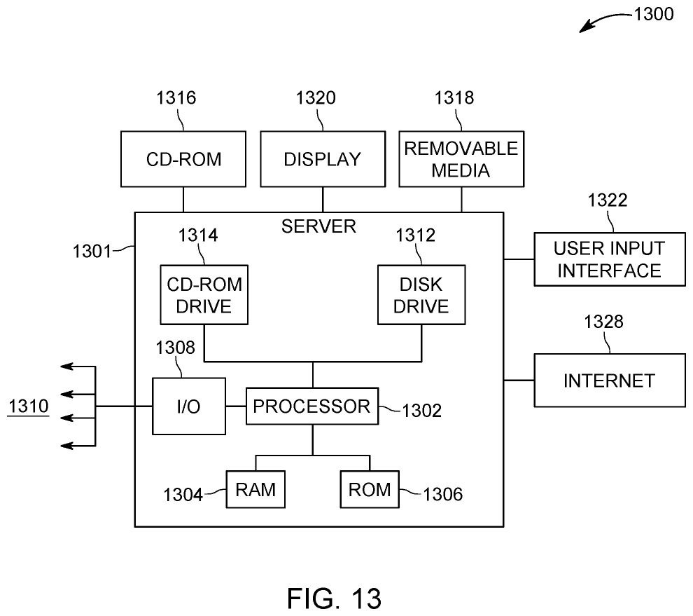

The above-discussed procedures and methods may be implemented in a computing device as illustrated in FIG. 13. Hardware, firmware, software or a combination thereof may be used to perform the various steps and operations described herein.

Exemplary computing device 1300 suitable for performing the activities described in the above embodiments may include a server 1301. Such a server 1301 may include a central processor (CPU) 1302 coupled to a random access memory (RAM) 1304 and to a read-only memory (ROM) 1306. ROM 1306 may also be other types of storage media to store programs, such as programmable ROM (PROM), erasable PROM (EPROM), etc. Processor 1302 may communicate with other internal and external components through input/output (I/O) circuitry 1308 and bussing 1310 to provide control signals and the like. Processor 1302 carries out a variety of functions as are known in the art, as dictated by software and/or firmware instructions.

Server 1301 may also include one or more data storage devices, including hard drives 1312, CD-ROM drives 1314 and other hardware capable of reading and/or storing information, such as DVD, etc. In one embodiment, software for carrying out the above-discussed steps may be stored and distributed on a CD-ROM or DVD 1316, a USB storage device 1318 or other form of media capable of portably storing information. These storage media may be inserted into, and read by, devices such as CD-ROM drive 1314, disk drive 1312, etc. Server 1301 may be coupled to a display 1320, which may be any type of known display or presentation screen, such as LCD, plasma display, cathode ray tube (CRT), etc. A user input interface 1322 is provided, including one or more user interface mechanisms such as a mouse, keyboard, microphone, touchpad, touch screen, voice-recognition system, etc.

Server 1301 may be coupled to other devices, genome sequencing device, detectors, etc. The server may be part of a larger network configuration as in a global area network (GAN) such as the Internet 1328, which allows ultimate connection to various landline and/or mobile computing devices.

The computing device 1300 may be configured to implement the deep neural network model 100 and thus, the processor 1302 provide a medium for an input layer 104, which is configured to receive a known genome sequence and plural convolutional neural networks, CNN, layers 110-I, 112, each connected to the input layer 104, and the CNN layers are configured to train with a current negative set obtained from the known genome sequence. The memory 1304, 1306 is connected to the processor 1302 and is configured to record false positive sets when the deep neural network model is applied to the known genome sequence. The processor is further configured to select a subset of the new false positive sets, to update the current negative set with the new false positive sets, and to repeat the steps of training, applying, selecting and updating until a number of the new false positive sets is smaller than a given threshold.

The processor further has a softmax layer connected to the CNN layers and the softmax layer is configured to calculate a score for plural sets of the known genome sequence. In one embodiment, each of the CNN layers has a filter and no two filters have the same size.

The disclosed embodiments provide a model and training method for the model for finding a transcription start site for a promoter in the human genome. It should be understood that this description is not intended to limit the invention. On the contrary, the embodiments are intended to cover alternatives, modifications and equivalents, which are included in the spirit and scope of the invention as defined by the appended claims. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

### REFERENCES

[1] Yehuda M Danino, Dan Even, Diana Ideses, and Tamar Juven-Gershon. The core promoter: At the heart of gene expression. Biochimica et biophysica acta, 1849:1116-1131, August 2015. ISSN 0006-3002. doi: 10.1016/j.bbagrm.2015.04.003.
[2] Long Vo Ngoc, California Jack Cassidy, Cassidy Yunjing Huang, Sascha H C Duttke, and James T Kadonaga. The human initiator is a distinct and abundant element that is precisely positioned in focused core promoters. Genes & development, 31:6-11, January 2017a. ISSN 1549-5477. doi: 10.1101/gad.293837.116.
[3] Ramzan Kh Umarov and Victor V Solovyev. Recognition of prokaryotic and eukaryotic promoters using convolutional deep learning neural networks. PloS one, 12:e0171410, 2017a. ISSN 1932-6203. doi: 10.1371/journal.pone.0171410.
[4] Diederik P Kingma and Jimmy Ba. Adam: A method for stochastic optimization. arXiv preprint arXiv:1412.6980, 2014.

1. A method for training a deep neural network model based on a known genome sequence, the method comprising:
receiving the known genome sequence;
training the deep neural network model with a current negative set obtained from the known genome sequence;
applying the deep neural network model to the known genome sequence and recording false positive sets;
selecting a subset of the new false positive sets;
updating the current negative set with the new false positive sets; and
repeating the steps of training, applying, selecting and updating until a number of the new false positive sets is smaller than a given threshold.

2. The method of claim 1, wherein the negative set includes a region of the known genome sequence that does not include a promoter, a positive set includes a region of the known genome sequence that includes a promoter, and a false positive set includes a region of the known genome sequence that does not include a promoter but is found by the deep neural network model to correspond to a promoter.

3. The method of claim 1, further comprising:
calculating a score for plural sets of the known genome sequence based on plural convolutional neural networks layers of the deep neural network model; and
selecting the subset of the new false positive sets based on a highest score of the plural sets.

4. The method of claim 3, wherein the score is calculated by a softmax layer, the softmax layer has two neurons, a first neuron which represents an input sequence being a promoter and a second neuron which represents an input sequence being a non-promoter.

5. The method of claim 4, wherein the score is given by a difference of pp and pnp, to which the unity is added, and a result is divided by 2.

6. The method of claim 1, wherein the step of updating further comprises:
removing a subset of the current negative set.

7. The method of claim 1, further comprising:
training the deep neural network model for promoters having a TATA box to obtain a TATA+ trained model;
training the deep neural network model for promoters not having a TATA box to obtain a TATA− trained model.

8. The method of claim 1, wherein the current negative set is originally randomly obtained from the known genome sequence.

9. A method for determining a transcription start site of a promoter in a genome sequence, the method comprising:
receiving a genome sequence;
training a deep neural network model based on an interactive and adaptive approach that updates a current negative set based on determined false positives;
applying the genome sequence to the deep neural network model; and
determining the transcription start site of the promoter in the genome sequence based on the updated current negative set.

10. The method of claim 9, wherein the training step comprises:
training the deep neural network model with a current negative set obtained from a known genome sequence;
applying the deep neural network model to the known genome sequence and recording false positive sets;
selecting a subset of the new false positive sets;
updating the current negative set with the new false positive sets; and
repeating the steps of training, applying, selecting and updating until a number of the new false positive sets is smaller than a given threshold.

11. The method of claim 10, wherein the negative set includes a region of the known genome sequence that does not include a promoter, a positive set includes a region of the known genome sequence that includes a promoter, and a false positive set includes a region of the known genome sequence that does not include a promoter but is found by the deep neural network model to correspond to a promoter.

12. The method of claim 10, further comprising:
calculating a score for plural sets of the known genome sequence based on plural convolutional neural networks layers of the deep neural network model; and
selecting the subset of the new false positive sets based on a highest score of the plural sets.

13. The method of claim 12, wherein the score is calculated by a softmax layer, the softmax layer has two neurons, a first neuron which represents an input sequence being a promoter and a second neuron which represents an input sequence being a non-promoter.

14. The method of claim 13, wherein the score is given by a difference of pp and pnp, to which the unity is added, and a result is divided by 2.

15. The method of claim 10, wherein the step of updating further comprises:
removing a subset of the current negative set.

16. The method of claim 10, further comprising:
training the deep neural network model for promoters having a TATA box to obtain a TATA+ trained model;
predicting promoters having the TATA box by using the TATA+ trained model;
training the deep neural network model for promoters not having a TATA box to obtain a TATA− trained model;
predicting promoters not having the TATA box by using the TATA− trained model; and
combining the promoters having the TATA box with the promoters not having the TATA box to determine the transcription start site of the promoter in the genome sequence.

17. A computing device that implements a deep neural network model, which comprises:
a processor having an input layer configured to receive a known genome sequence and plural convolutional neural networks, CNN, layers, each connected to the input layer, and configured to train with a current negative set obtained from the known genome sequence;
a memory connected to the processor and configured to record false positive sets when the deep neural network model is applied to the known genome sequence; and
the processor being configured to select a subset of the new false positive sets, to update the current negative set with the new false positive sets, and to repeat the steps of training, applying, selecting and updating until a number of the new false positive sets is smaller than a given threshold.

18. The computing device of claim 17, wherein the negative set includes a region of the known genome sequence that does not include a promoter, a positive set includes a region of the known genome sequence that includes a promoter, and a false positive set includes a region of the known genome sequence that does not include a promoter but is found by the deep neural network model to correspond to a promoter.

19. The computing device of claim 17, wherein the processor further has a softmax layer connected to the CNN layers and the softmax layer is configured to calculate a score for plural sets of the known genome sequence.

20. The computing device of claim 17, wherein each of the CNN layers has a filter and no two filters have the same size.
